(19) United States
(12) Patent Application Publication
(10) Pub. No.: US 2020/0276418 A1
(43) Pub. Date: Sep. 3, 2020

(54) VARIABLE STIFFNESS GUIDE WIRE

(71) Applicant: W. L. Gore & Associates, Inc., Newark, DE (US)

(72) Inventors: Michael T. Wiehn, Flagstaff, AZ (US); Martin J. Sector, Phoenix, AZ (US)

(21) Appl. No.: 16/647,712

(22) PCT Filed: Sep. 14, 2018
(86) PCT No.: PCT/US2018/051049

§ 371 Date: Mar. 16, 2020

Related U.S. Application Data
(60) Provisional application No. 62/558,402, filed on Sep. 14, 2017.

(51) Int. Cl. A61M 25/09

(52) U.S. Cl. CPC A61M 25/09; A61M 2025/09083; A61M 2205/0233; A61M 2025/09158; A61M 2025/09141

(57) ABSTRACT

Medical systems, including guide wires and guide wire assemblies 1100, 2100, 3100 that are configured such that a flexibility of the guide wire can be changed or modified. In various embodiments, the flexibility of the guide wire can be changed or modified in-situ. The guide wires and/or guide wire assemblies of the disclosed medical systems may include materials having material properties that can be modified in association with exposing one or more portions of the medical system, such as one or more components of the guide wire or guide wire assembly to electric current.

### CROSS-REFERENCE TO RELATED APPLICATION

This application claims the benefit of Provisional Application No. 62/558,402, filed Sep. 14, 2017, which is incorporated herein by reference in its entirety for all purposes.

### BACKGROUND

The present disclosure is related to intravascular delivery devices and more particularly to guide wires configured to include one or more selectively variable mechanical properties, such as flexibility.

Physicians generally require the use of one or more guide wires to gain access to and deliver therapeutic and/or diagnostic devices to intravascular regions requiring treatment within the body. A relatively flexible guide wire is selected and utilized to facilitate navigation through tortuous vasculature. However, relatively stiff guide wires are typically utilized during device delivery and deployment because they provide the requisite support needed for proper delivery as well as stability during deployment. Thus, in some cases, a combination of guide wires is required to complete a procedure.

Abdominal aortic aneurysmal (“AAA”) repair is one of many exemplary procedures where multiple different guide wires are utilized during the course of a medical procedure. For instance, in some AAA cases, three (3) or more different guide wires are utilized during the procedure. A first flexible guide wire is used to initially navigate the tortuous structure of the vasculature in order to access the treatment site within the aorta. Thereafter, a catheter may be advanced over the first flexible guide wire. The first flexible guide wire is subsequently removed and replaced with a stiffer guide wire that is suitable for deploying a medical device, such as a stent or stent graft. In some cases involving the deployment of a bifurcated stent-graft, a third guide wire is used to cannulate the contralateral leg of the bifurcated stent-graft. In some cases, the first flexible guide wire used to initially navigate the tortuous structure of the vasculature lacks the requisite stability needed to facilitate proper deployment.

### SUMMARY

According to one example, (“Example 1”), a medical system includes a guide wire assembly that includes a guide wire member including an alloy and having a flexibility that is configured to change when exposed to an electrical current; and an insulation material surrounding at least a portion of the guide wire member. The medical system further includes a controller electrically coupled to the guide wire assembly and configured to cause an electrical current to be selectively supplied to the guide wire assembly such that the flexibility of the guide wire assembly changes in response to an exposure to the electrical current.

According to one example, (“Example 2”), a medical system includes a guide wire assembly configured to transition between a first configuration and a second configuration, wherein a flexibility of the guide wire assembly in the first configuration exceeds the flexibility of the guide wire assembly in the second configuration, the guide wire assembly including: a guide wire member including an alloy; and an insulation material surrounding at least a portion of the guide wire member. The medical system further includes a controller electrically coupled to the guide wire assembly and configured to cause an electrical current to be selectively supplied to the guide wire assembly to cause the guide wire assembly to transition between the first and second configurations.

According to another example, (“Example 3”) further to any of the preceding Examples, the alloy including a phase-changeable alloy.

According to another example, (“Example 4”) further to any of the preceding Examples, the alloy including nitinol.

According to another example, (“Example 5”) further to any of the preceding Examples, the guide wire member including a first core member and a second core member coupled to the first core member the first core member including the alloy such that the guide wire member is configured to change its flexibility when exposed to the electrical current, wherein the first and second core members are coupled to one another at respective first ends of the first and second core members, and wherein respective second ends of the first and second core members are coupled with the controller.

According to another example, (“Example 6”) further Example 5, one or more of the first and second core members extend generally linearly along a longitudinal axis of the guide wire assembly when exposed to electrical current.

According to another example, (“Example 7”) further to any of Examples 5 or 6, wherein the first and second core members are aligned parallel to one another.

According to another example, (“Example 8”) further Example 5, wherein the second core member is helically coiled about the first core member.

According to another example, (“Example 9”) further to Example 5, wherein the first and second core members are each helically wound about a longitudinal axis of the guide wire assembly.

According to another example, (“Example 10”) further to any of Examples 5 to 9, wherein the first core member and the second core member are formed from different materials.

According to another example, (“Example 11”) further to any of Examples 5 to 10, wherein the first core member and the second core member are formed from different alloys.

According to another example, (“Example 12”) further to any of the preceding Examples, the guide wire assembly varies in flexibility to allow it to function for at least two of the following guide wire purposes: tracking, deployment, and cannulation.

According to another example, (“Example 13”) further to any of the preceding Examples, the controller is operable to cause a current to flow through a first portion of the guide wire member and wherein the insulation material surrounds the first portion.

According to another example, (“Example 14”) a method of making a medical system includes providing a guide wire member including an alloy; disposing an insulation material about at least a portion of the guide wire member to define a guide wire assembly, the guide wire assembly having a flexibility that is configured to change when exposed to an electrical current; and electrically coupling a controller to the guide wire assembly such that the controller is operable to cause an electrical current to be selectively supplied to the guide wire assembly such that the flexibility of the guide wire assembly changes in response to an exposure to the electrical current.

According to another example, (“Example 15”) a method of treatment includes: providing a guide wire assembly that includes a guide wire member having an alloy and having a flexibility that is configured to change when exposed to an electrical current; and an insulation material surrounding at least a portion of the guide wire member. The method further includes electrically coupling a controller to the guide wire assembly such that the controller is operable to cause an electrical current to be selectively supplied to the guide wire assembly; and causing the controller to supply a first electrical current to the guide wire assembly to cause the flexibility of the guide wire assembly to change from a first flexibility to a second flexibility, wherein the first flexibility exceeds the second flexibility.

While multiple embodiments are disclosed, still other embodiments will become apparent to those skilled in the art from the following detailed description, which shows and describes illustrative examples. Accordingly, the drawings and detailed description are to be regarded as illustrative in nature and not restrictive.

### BRIEF DESCRIPTION OF THE DRAWINGS

The accompanying drawings are included to provide a further understanding of inventive embodiments of the disclosure and are incorporated in and constitute a part of this specification, illustrate examples, and together with the description serve to explain inventive principles of the disclosure.

FIG. 1 is an illustration of a variable stiffness guide wire, according to some embodiments.

FIG. 2 is an illustration of a cross section of the variable stiffness guide wire illustrated in FIG. 1 taken along line 2-2, according to some embodiments.

FIG. 3 is an illustration of a cross section of a variable stiffness guide wire, according to some embodiments.

FIG. 4 is an illustration of a cross section of a variable stiffness guide wire, according to some embodiments.

### DETAILED DESCRIPTION

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting. In describing various examples, the term proximal is used to denote a position along the exemplary device proximate to or alternatively nearest to the user or operator of the device. Proximal may also be referred to as trailing. The term distal is used to denote a position along an exemplary device farthest or farther from the user or operator of the device. Distal may also be referred to as leading.

Various aspects of the present disclosure are directed toward guide wires and the like for utilization during medical procedures to locate treatment regions within a patient's vasculature and/or facilitate the delivery and deployment of one or more medical devices to the treatment region within the vasculature. More specifically, the present disclosure relates to guide wire devices and systems, and methods for using such guide wire devices and systems.

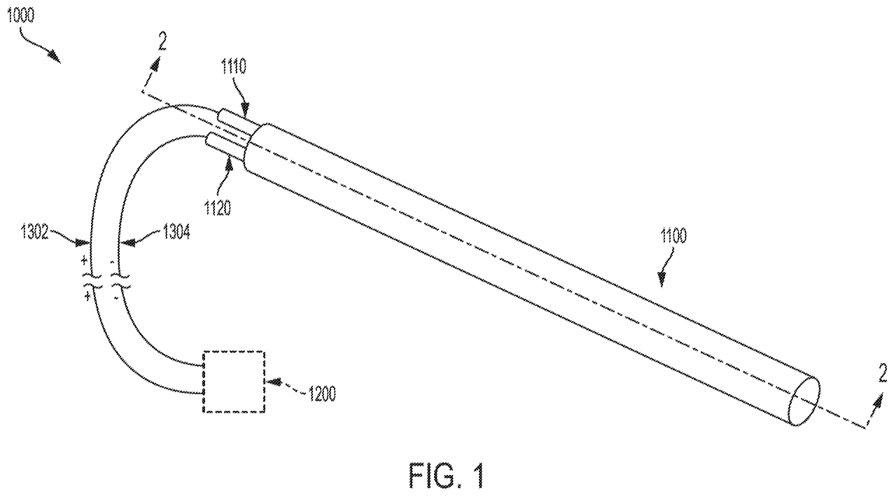

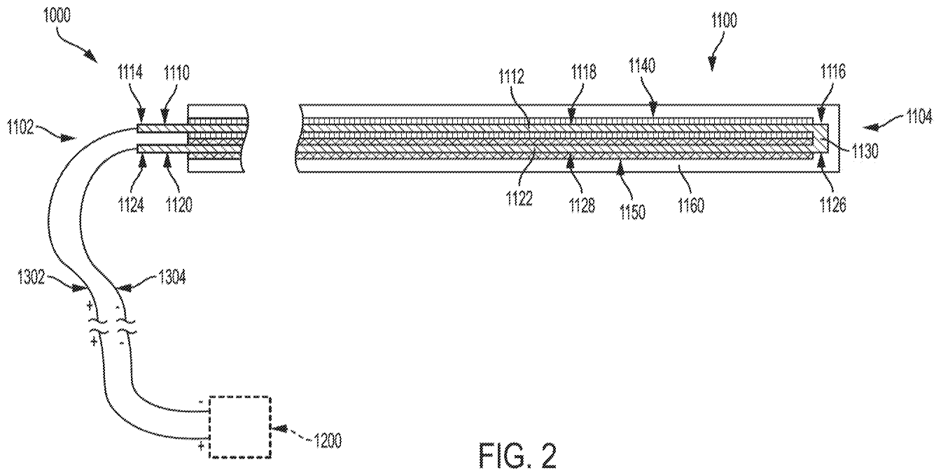

In various embodiments, a guide wire system 1000 as illustrated in FIGS. 1 and 2 includes a guide wire assembly 1100 and a controller 1200 electrically coupled to the guide wire assembly 1100. FIG. 2 is a cross sectional view of the guide wire assembly of 1100 illustrated in FIG. 1 taken along lines 2-2. The guide wire assembly 1100 is generally cylindrically shaped having a generally circular cross-section and includes an elongate shaft having a proximal end 1102 and a distal end 1104. Those of skill in the art will appreciate that the guide wire assembly 1100 may include any suitable cross sectional shape. For example, the cross sectional shape may have curved aspects, linear aspects, or combinations thereof (e.g., ovular or polygonal) without departing from the spirit or scope of the application. Likewise, while the cross-section of the guide wire assembly 1100 illustrated in FIG. 2 is generally uniform along its length, it should be appreciated that the cross section may vary without departing from the spirit or scope of the inventive concepts discussed herein. For instance, in various examples, the cross section of the guide wire assembly may taper longitudinally. In such examples, a distal end may have a different cross sectional area than a proximal end and/or an intermediate portion situated between the proximal and distal ends.

In various examples, the guide wire assembly 1100 is generally insulated (e.g., electrically and/or thermally) and includes a plurality of core members, such as first core member 1110 and second core member 1120. As discussed in greater detail below, a flexibility or stiffness of the guide wire assembly 1100 can be changed or adjusted during operation (e.g., in-situ) by inducing a current through the first and second core members 1110 and 1120 of the guide wire assembly 1100. In various examples, the flexibility or stiffness of one or more of the first and second core members 1110 and 1120 of the guide wire assembly 1100 can be controlled through operation of the controller 1200. Such a configuration provides that, unlike conventional designs, the same guide wire assembly can be utilized during an operation to both locate a treatment region within a patient's vasculature and facilitate the delivery and deployment of one or more medical devices to the treatment region within the vasculature. For instance, as explained in greater detail below, after locating a target treatment region within a patient's vasculature, a flexibility of the guide wire can be modified or adjusted such that a medical device can be delivered and deployed over the guide wire.

As mentioned above, the guide wire assembly 1100 includes a plurality of core members, including a first core member 1110 and a second core member 1120. In various examples, one or more of the first and second core members 1110 and 1120 include, or otherwise formed from, a material that changes one or more physical properties when subjected to stimulation from an exterior energy source, such as an electrical power source. Thus, in various examples, the first and second core members 1110 and 1120 include a material that is electrically conductive. Suitable non-limiting exemplary materials include, but are not limited to, alloys and phase changeable alloys such as nickel-titanium alloys like nitinol (NiTi), doped nickel-titanium alloys, gold cadmium alloys, silver cadmium alloys, copper alloys, magnesium alloys, cobalt alloys, and the like. In some examples, polymeric material can be melted to achieve similar phase changeable properties, as those of skill in the art will appreciate. In some examples, these materials are shape settable in that they can transition between a first configuration and a second different configuration upon being heated beyond a critical temperature (e.g., a temperature at which the material undergoes a transition between martensitic and austenitic states), as those of skill in the art will appreciate. Generally, the first configuration in which the material is compliant or relatively flexible in comparison to the second configuration wherein the material is more stiff or less flexible. Relative stiffness or flexibility can be measured using a standard three-point bending test or any other test recognized by those in the field as suitable for a particular application. For example, ASTM D790 refers to possible non-limiting test methods for flexural properties of unreinforced and reinforced plastics and electrical insulating materials that could be used to measure relative stiffness and flexibility.

In various examples, the first and second core members 1110 and 1120 generally include a body having proximal and distal ends. For example, as shown in FIG. 2, a first core member 1110 includes a body 1112, a proximal end 1114, and a distal end 1116. The first core member 1110 additionally includes an intermediate portion 1118 that is situated between the proximal and distal ends 1114 and 1116. Likewise, as shown in FIG. 2, a second core member 1120 includes a body 1122, a proximal end 1124, a distal end 1126, and an intermediate portion 1128 situated between the proximal and distal ends 1124 and 1126.

As mentioned above, in various embodiments, a current is induced through the guide wire assembly 1100 to adjust a flexibility of the guide wire assembly 1100. In various examples, the first and second core members 1110 and 1120 of the guide wire assembly 1100 are electrically coupled together to form a circuit through which current can be passed or otherwise induced. While the first and second core members 1110 and 1120 may be coupled together at one or more of a plurality of locations along their length, in various examples, the first and second core members 1110 and 1120 are electrically coupled together at an end opposite the ends to which the electrical leads are coupled. For example, as shown in FIG. 2, distal ends 1116 and 1126 of the first and second core members 1110 and 1120, respectively, are electrically coupled together at joint 1130. That is, a joint 1130 is established where the distal ends 1116 and 1126 of the first and second core members 1110 and 1120 are electrically coupled together. Suitable non-limiting exemplary mechanisms and methods for electrically coupling the first and second core members 1110 and 1120 together include welding, soldering, adhering, or banding together with one or more fasteners including electrically conductive fasteners as those of skill should appreciate.

In various examples, the passage of current through the core members generates heat, which causes a change in one or more physical properties of the core members (e.g., flexibility), as discussed in greater detail below. In various examples, such heat generation is due in part to the resistance of the material through which the current is passing.

While the core members may be electrically coupled together at one or more portions or points along their length, in various examples, the core members may be additionally or alternatively electrically isolated from one another at one or more locations or regions along their lengths. Such a construction provides that the current passing through the core members follows a predetermined path, which facilitates a guide wire assembly 1100 having a flexibility and structure that can be selectively controlled during its use in association with a medical procedure.

In various examples, a point or region of a core member is electrically isolated by disposing or surrounding an insulative material about designated portions of the core member. In some examples, the insulative material may be in the form of a sleeve that is disposed about the core member or alternatively a sleeve within which the core member is inserted. In other examples, the insulative material may be in the form of a material that is wrapped about the core member. For instance, an insulative material in the form of a tape may be wrapped (e.g., helically or longitudinally) about the core member. In other examples, the insulative material may be disposed about the core member by way of one or more dipping processes. Similarly, in some examples, the insulative material may be disposed about the core member by way of one or more spray processes. In some examples, after an insulative material has been applied to a core member, one or more processes may be utilized to remove portions of the insulative material from one or more designated regions, areas, or portions of the core member to expose such designated regions, areas, or portions. It should be appreciated that an insulative material may be disposed about the core member such that the core member is entirely insulated (e.g., electrically, thermally, or both). In some examples, an insulative material may be disposed about the core member such that the conductive elements of the guide wire assembly are prevented from electrically interacting with the surrounding body environment including the body tissue. Likewise, in some example, an insulative material may be disposed about the core member such that the surrounding body environment including the body tissue is protected against any damaging amounts of thermal energy generated by the guide wire assembly. Thus, in various examples, the insulative material is disposed about the guide wire assembly such that the surrounding body environment is not otherwise exposed to electrical or thermal elements that may cause damage.

Those of skill in the art should appreciate that an insulative material may be disposed about the core members individually or collectively. For instance, in some examples, each core member includes an insulative material individually disposed thereabout. In some other examples, an insulative material is disposed about a plurality of core members. For example, a plurality of core members may be collected or bunched together and an insulative material is disposed about the collection or bunch.

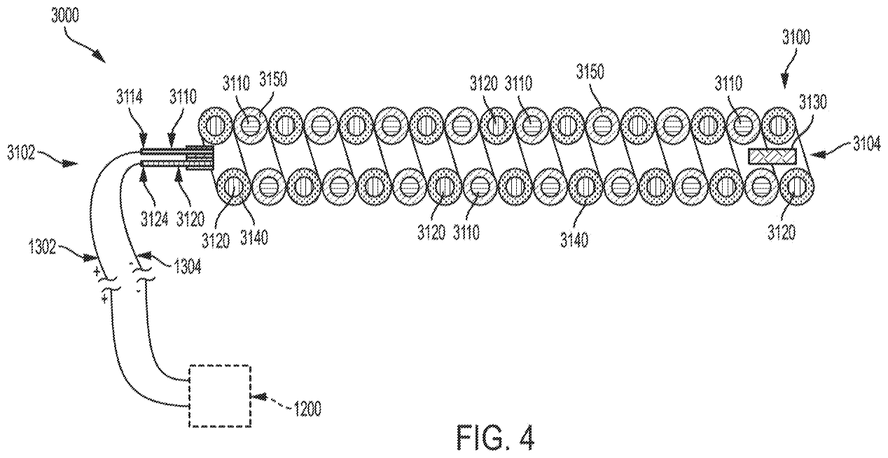

In some examples, an insulative material or layer is disposed about one or more, but less than all, of the core members. Thus, in some examples, the guide wire assembly is configured such that at least one core member of the guide wire assembly does not have an insulative material disposed thereabout to independently isolate the core member from the other core members of the guide wire assembly. However, in some such examples, the insulative material disposed about the other core members operates to isolate the core members from one another (see e.g., FIG. 4). Thus, in some examples, an insulative layer disposed about a first core member operates to electrically isolate the first core member and a second adjacently situated and exposed core member along the length of the insulative layer. Additionally, those of skill should appreciate that the insulative layers additionally operate to protect surrounding tissue from damage due to exposure to heat and/or electric current.

Referring again to FIG. 2, as shown, the first and second core members 1110 and 1120 of the guide wire assembly 1100 each include an insulative material disposed thereabout. For example, an insulative layer 1140 is disposed about the first core member 1110 and an insulative layer 1150 is disposed about the second core member 1120. As shown, the distal and proximal ends of the first and second core members 1110 and 1120 are exposed or not otherwise covered by the insulative layers 1140 and 1150. That is, as shown in the illustrated example of FIG. 2, the insulative layers 1140 and 1150 are each disposed about only a portion of their respective first and second core members 1110 and 1120.

Specifically, as shown, insulative layer 1150 is disposed about the second core member 1120 such that the proximal and distal ends 1124 and 1126 of second core member 1120 remain exposed or uncovered. Likewise, as shown, insulative layer 1140 is disposed about core member 1110 such that the proximal and distal ends 1114 and 1116 of core member 1110 remain exposed or uncovered. Thus, in various examples, an insulative layer may be applied to a core member of a guide wire assembly such that one or more portions remain uncovered or exposed. While the proximal and distal ends of the core members illustrated in FIG. 2 remain exposed or uncovered, those of skill should appreciate that the insulative layer may be applied to a core member of the guide wire assembly such that one or more regions of the core members other than the proximal and distal ends (e.g., intermediate portions, or one or more discrete portions thereof) may be additionally or alternatively exposed or uncovered.

In various examples, as mentioned above, the guide wire assembly may additionally or alternatively include one or more insulative layers disposed about the plurality of core members. That is, one or more insulative layers may be disposed about the plurality of core members in addition to or as an alternative to any insulative layers that are individually disposed about the core members of the guide wire assembly. For example, as shown in FIG. 2, an insulative layer 1160 is disposed about the first and second core members 1110 and 1120 in addition to the insulative layers 1140 and 1150 that are individually disposed about the first and second core members 1110 and 1120, respectively. In various examples, insulative layer 1160 forms or otherwise defines an exterior of the guide wire assembly 1100. In some examples, the insulative layer 1160 is disposed about the distal ends of the core members such that insulative layer 1160 defines the distal end 1104 of the guide wire assembly 1100.

However, those of skill in the art should appreciate that other examples are envisioned where one or more other features are disposed about the distal ends of the core members. For instance, one or more covers or tips may be coupled to, or otherwise disposed about, the distal ends of the core members. Likewise, embodiments are also envisioned where the distal ends of the core members remain uncovered or otherwise exposed.

In some examples, the core members may be electrically coupled together (e.g., short circuited) at some point proximal to the distal ends thereof. That is, in some examples, the core members are coupled together such that the core members (and thus the guide wire assembly) includes a portion proximal to the coupling and a portion distal to the coupling. In some examples, current does not generally flow through the portion of the core members extending distal to the coupling. Such configurations provide for a guide wire assembly wherein one or more portions of the core member extending distal to the coupling are more compliant or otherwise not as stiff as one or more portions more proximate to the coupling and/or more proximal thereto. For instance, in some examples, the portion(s) of the core member(s) extending distal to the coupling have a temperature gradient thereacross resulting in a stiffness gradient thereacross wherein more distal portions are less stiff than more proximal portions.

In various examples, the insulative materials or layers discussed herein may include expanded polytetrafluoroethylene (ePTFE), fluorinated ethylene propylene (FEP), or any other suitable polymeric material. In some examples, the polymeric material includes, or is otherwise formed of, one or more layers, sheets, or films of polymeric material. Other non-limiting exemplary polymeric materials include, but are not limited to, polytetrafluoroethylene (PTFE), polyurethane, polysulfone, polyvinylidene fluorine (PVDF), polyhexafluoropropylene (PHFP), perfluoroalkoxy polymer (PFA), polyolefin, and acrylic copolymers. These materials can be in sheet, film, knitted or woven (e.g., fiber), or non-woven porous forms. In some examples, these materials are spray-coated onto a substrate or directly coated onto one or more of the core members or a material surrounding the core members. In some examples, the polymeric material is formed from a plurality of layers or sheets of polymeric material. In some such examples, the layers or sheets are laminated or otherwise mechanically coupled together, such as by way of heat treatment and/or high pressure compression and/or adhesives and/or other laminating methods known by those of skill in the art. Non-limiting examples of applying an insulation layer to a core member include helical wrapping, spray coating, dip coating, longitudinal wrapping, and the like, application through a polymer extrusion process, or a continuous barrier (controlled grounding).

As mentioned above, in various embodiments, the guide wire system includes a controller 1200 that is electrically coupled to the guide wire assembly 1100. In some embodiments, the controller 1200 operates to direct and control the delivery and/or flow of current to the guide wire assembly 1100. In some examples, the controller 1200 includes, or is otherwise electrically coupled with, a power source that is configured to deliver, or otherwise induce a current through, the guide wire assembly 1100. The power source may be integral with the system or may be externally coupleable and may include a conventional power supply with conventional control circuitry to provide a constant or modulated AC or DC signal. Various non-limiting examples of the applied current include a steady current, pulsing current, and sinusoidal current. In some examples, the controller further includes, or is otherwise electrically coupled to, an electronic regulator that operates to condition and control the electrical signal delivered to the guide wire assembly 1100. In various examples, the electronic regulator operates to increase and/or decrease resistance, and/or adjust pulse frequency, and/or increase and/or decrease current, and/or adjust amplitude.

As mentioned above, the controller 1200 is electrically coupled to the guide wire assembly 1100. In some examples, one or more electrical leads are situated between and electrically couple the controller 1200 to the guide wire assembly 1100. For example, as illustrated in FIG. 2, electrical leads 1302 and 1304 are situated between and electrically couple the controller 1200 to the guide wire assembly 1100. In various examples, the electrical leads include any lead suitable for delivering current to the guide wire assembly 1100. In various examples, the electrical leads are integral to the guide wire assembly 1100 in that the electrical leads are designed for single use as those of skill in the art will appreciate. In other examples, the electrical leads may be integral to the controller or may be otherwise configured for repeated use as those of skill in the art will appreciate. In yet other examples, the electrical lead components of the guide wire system 1000 are independent of the guide wire assembly 1100 and the controller 1200. In various examples, the leads can be temporarily disconnected from one or more components of the system such that medical devices (e.g., catheters, stents, grafts, stent-grafts, etc.) can be loaded onto and subsequently delivered and deployed over the guide wire assembly 1100. In some examples, current is applied to one or more of the core members during deployment of the medical device.

In various examples, the electrical leads are coupled to the guide wire assembly such that the core members of the guide wire assembly are electrically coupled to the controller, as discussed above. As illustrated in FIG. 2, electrical lead 1302 is situated between the controller 1200 and the guide wire assembly 1100 and electrically coupled to an exposed portion of the proximal end 1114 of the core member 1110 and a positive terminal of the controller 1200. Similarly, as shown in FIG. 2, electrical lead 1304 is situated between the controller 1200 and the guide wire assembly 1100 and electrically coupled to an exposed portion of the proximal end 1124 of the second core member 1120 and a negative terminal of the controller 1200.

While the proximal ends 1114 and 1124 of the first and second core members 1110 and 1120 are illustrated as being exposed and coupled to leads 1302 and 1304, respectively, in various examples, the proximal ends of the core members may be covered, concealed, or not otherwise exposed. For instance, in some examples, the proximal end of the guide wire assembly includes one or more terminals to which the electrical leads can be coupled. In some such examples, the terminals are electrically coupled to the corresponding core members of the guide wire assembly as those of skill will appreciate. In various embodiments, such a configuration provides that a potential or voltage may be drawn across the proximal ends of the core members of the guide wire assembly such that a current flows therethrough. In the specific example illustrated in FIG. 2, current is induced across the proximal ends 1114 and 1124 of the first and second core members 1110 and 1120 such that, within the guide wire assembly 1100, the current generally flows from the negative terminal proximal end 1124 of second core member 1120, through second core member 1120, through the joint 1130 between the first and second core members 1110 and 1120, through core member 1110, and to the positive terminal proximal end 1114 of the core member 1110.

In various examples, as current flows through the core members of the guide wire assembly, a temperature of the core members increases due to the resistive nature of the material of the core members. In these examples, the temperature of the core members generally increases in association with an increase in the current flowing through the core members (e.g., as a result of an increase in voltage potential drawn across the distal ends of the core members). As discussed in greater detail below, upon reaching a designated temperature, one or more of the core members undergoes a physical change such that a flexibility of the core member changes along its length or along a portion of its length. In various examples, this change in flexibility of the core member results in a change in flexibility of the guide wire assembly.

As mentioned above, in various examples, the core members of the guide wire assembly include alloys and phase changeable alloys such as nitinol (NiTi). As explained above, these core members are generally configured such that upon reaching a designated temperature, one or more properties of the material changes, causing a flexibility or stiffness of the core member to change. Specifically, upon heating a core member beyond a designated temperature, the core member loses flexibility and increases in stiffness. In various examples, in addition to losing flexibility and increasing in stiffness, the core member is predisposed to adopt a particular geometry. Those of skill in the art should appreciate that the core member may be predisposed to adopt virtually any desired geometry upon heating beyond the designated temperature.

Referring again to the guide wire assembly 1100 illustrated in FIG. 2, the first and second core members 1110 and 1120 are generally situated adjacent and parallel to one another and generally parallel to a longitudinal axis of the guide wire assembly 1100. In this illustrated example, each of the first and second core members 1110 and 1120 are predisposed to adopt a linear shape and extend along the longitudinal axis of the guide wire assembly 1100 (as shown) when heated. Thus, when a temperature of the first and second core members 1110 and 1120 is elevated above a designated or critical temperature, each of the first and second core members 1110 and 1120 are extended linearly along the longitudinal axis of the guide wire assembly 1100 (as shown) and stiffen (or lose flexibility). Accordingly, one or more of the core members (and thus the guide wire assembly) is configured to transition between a first configuration and a second different configuration upon being heated beyond a designated temperature, wherein in the first configuration the core member (and thus the guide wire assembly) is compliant or relatively flexible in comparison to the second configuration, wherein the core member (and thus the guide wire assembly) is more stiff or less flexible. It should also be appreciated that the core member may also change shape when transitioning between the first and second configurations (e.g., between martensitic and austenitic states).

While the above-referenced example illustrated in FIG. 2 includes first and second core members 1110 and 1120, wherein the first and the second core members 1110 and 1120 each become relatively less flexible and more stiff when transitioning between the first and second configuration, those of skill in the art should appreciate that, in some alternative examples, only one of the core members (or less than all of the core members) is configured to become relatively less flexible and more stiff when transitioning between the first and second configurations. For example, as discussed further below, one or more of the core members may be configured to maintain its flexibility and shape when its temperature is elevated above the designated or critical temperature. As explained below, this may be a result of a specific heat treatment or the core member may be formed from a non-phase changeable alloy or material that does not otherwise increase its rigidity as its temperature is elevated.

Additionally, while the illustrated example of FIG. 2 includes a plurality of core members that are longitudinally aligned and configured to extend linearly along the longitudinal axis of the guide wire assembly 1100 (as shown) and stiffen (or lose flexibility) as their associated temperature is elevated, those of skill in the art should appreciate that various alternative core member configurations are contemplated and fall within the scope of the inventive concepts addressed in the instant disclosure.

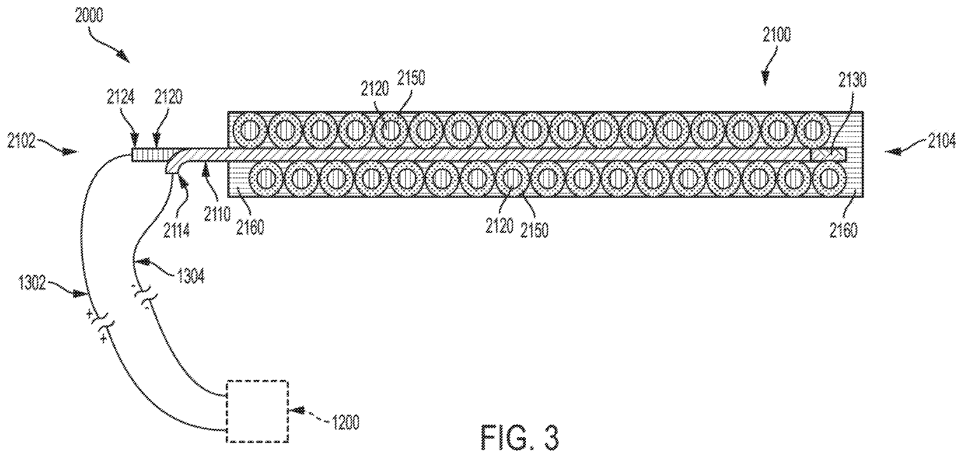

For example, with reference now to FIG. 3, a guide wire system 2000 is illustrated as including a guide wire assembly 2100 including a first core member 2110 and a second core member 2120 helically wrapped about the first core member 2110. In some examples, the guide wire system 2000 includes a controller 1200 electrically coupled to the guide wire assembly 2100, as shown. As discussed above, in some examples, the controller 1200 includes, or is otherwise electrically coupled with, a power source that is configured to deliver or otherwise induce a current through a guide wire assembly, such as guide wire assembly 2100. As shown in FIG. 3, the controller 1200 is coupled to the guide wire assembly 2100 via leads 1302 and 1304.

The cross-sectional view in FIG. 3 of the guide wire assembly 2100 illustrates the guide wire assembly 2100 as including the first core member 2110 and the helically wound second core member 2120 coupled to one another at their distal ends to form a joint 2130. Like the guide wire assembly 1100, the guide wire assembly 2100 is generally cylindrically shaped having a generally circular cross section and includes an elongate shaft having a proximal end 2102 and a distal end 2104. As shown, the joint 2130 is proximate the distal end 2104 of the guide wire assembly 2100. In various examples, joint 2130 is constructed in the same or similar manner as joint 1130 discussed above.

The first core member 2110 is similar to the first core member 1110 of the guide wire assembly 1100 in that the first core member 2110 includes a body having a proximal end 2114 and a distal end, and an intermediate portion situated between the proximal and distal ends. Similarly, like the second core member 1120 of the guide wire assembly 1100, the second core member 2120 includes a body having a proximal end 2124 and a distal end (not shown), and an intermediate portion situated between the proximal and distal ends.

Additionally, like the first core member 1110 of the guide wire assembly 1100 discussed above, the first core member 2110 of guide wire assembly 2100 is predisposed to adopt a linear shape and extend along the longitudinal axis of the guide wire assembly 2100 (as shown). Thus, when a temperature of the first core member 2110 is elevated above a designated or critical temperature, the first core member 2110 is configured to extend linearly along the longitudinal axis of the guide wire assembly 2100 (as shown) and stiffen (or lose flexibility).

However, as shown, the second core member 2120 is helically wound about the first core member 2110. That is, while the first and second core members 1110 and 1120 of the guide wire assembly 1100 are generally the same shape, size, and length, in the illustrated example of FIG. 3, because the second core member 2120 is helically wound about a portion of the first core member 2110 and extends generally the same length along the longitudinal axis of the guide wire assembly 2100 as the first core member 2110, the second core member 2120 is longer or has a longer axial length than the first core member 2110 (as measured along the longitudinal axis of the second core member 2120). In various examples, the second core member 2120 is predisposed to maintain its helical winding configuration about first core member 2110 when its temperature is elevated above a designated or critical temperature. For instance, in some examples, the second core member 2120 is configured such that when a temperature of the second core member 2120 is elevated above a designated or critical temperature, the second core member 2120 stiffens or loses flexibility, but is predisposed to adopt or otherwise maintain its helical winding shape about the first core member 2110.

In other examples, a core member may be heat treated in a manner that destroys its shape memory properties as those of skill in the art will appreciate. That is, in some examples, a member may be heat treated such that it is not predisposed to stiffen or lose flexibility as its temperature is elevated, but rather generally maintains its stiffness or flexibility across the operating temperature range. In some examples, a portion of less than all of the core members may be subjected to such heat treatment such that a portion of less than all of the core members not predisposed to stiffen or lose flexibility as their temperatures are elevated, but rather generally maintain their stiffness or flexibility across the operating temperature range. Such a configuration provides that a guide wire assembly may be formed with a single core member having a first portion and a second portion, wherein the first portion is configured to stiffen and/or change shape upon the core member's temperature being elevated to or beyond a designated temperature, and wherein the second portion is configured to maintain its shape and flexibility upon the core member's temperature being elevated to or beyond the designated temperature.

In some examples with variable stiffness properties, the core member may include a proximal and distal end, and an intermediate portion between the proximal and distal ends. The proximal and distal ends of the core member may be situated proximate the proximal end of the guide wire assembly and the intermediate portion may be situated proximate the distal end of the guide wire assembly. In such a configuration, the first portion includes the portion between the proximal end and intermediate portion, and the second portion includes the portion between the distal end and intermediate portion. Those of skill in the art should appreciate that the core member may be configured such that the first portion (or alternatively the second portion) is configured to stiffen and/or change shape upon the core member's temperature being elevated to or beyond a designated temperature and such that the second portion (or alternatively the first portion) is configured to maintain its shape and flexibility upon the core member's temperature being elevated to or beyond the designated temperature.

In some examples, the second core member 2120 may be formed from a non-phase changeable alloy or material that does not otherwise increase its rigidity as its temperature is elevated. In these examples, despite not increasing in stiffness with an elevation in temperature, the second core member 2120 nevertheless operates with the first core member 2110 to complete a circuit such that current can be induced through the guide wire assembly 2100.

In some examples, the second core member 2120 is predisposed to adopt a linear shape and extend along the longitudinal axis of the guide wire assembly 2100 as its temperature is elevated above a designated or critical temperature. That is, although the second core member 2120 is helically wound about the first core member 2110, as current flows through the guide wire assembly 2100 and the temperature of the second core member 2120 is elevated above a designated or critical temperature, the second core member 2120 is predisposed to adopt a linear shape and extend along the longitudinal axis of the guide wire assembly 2100. In some examples, this expansion of the second core member 2120 induces the second core member 2120 to unwind helically and lengthen relative to the longitudinal axis of the guide wire assembly 2100. However, the joint 2130 where the first and second core members 2110 and 2120 are coupled together operates to constrain the second core member 2120 from elongating relative to the first core member 2110, which tensions the first core member 2110 and thus adds further stiffness to the guide wire assembly 2100 as those of skill will appreciate.

In a manner similar to or the same as that discussed above regarding the guide wire assembly 1100, an insulative layer 2150 is disposed about the second core member 2120 and an insulative layer 2160 is disposed about core members 2110 and 2120 in addition to any insulative layers that are individually disposed about the core members 2110 and 2120. In various examples, insulative layer 2160 forms or otherwise defines an exterior of the guide wire assembly 2100. In various examples, insulative layer 2150 is constructed and disposed about the second core member 2120 in a same or similar manner as insulative layer 1150 is disposed about second core member 1120, discussed above. However, as shown in FIG. 3, an insulative layer is not individually disposed about the first core member 2110 (see, e.g., the discussion above regarding the application of layers about the first and second core members 1110 and 1120). In some examples, the insulative layer 2150 disposed about the second core member 2120 electrically isolates core members 2110 and 2120 from one another.

While the second core member 2120 is illustrated in FIG. 3 as having generally constant helical windings, it should be appreciated that the second core member 2120 may be wound about the first core member 2110 with helical windings that vary in pitch along the length of the first core member 2110. In some examples, the helical windings generally progressively increase (or alternatively decrease) in pitch along the length of the first core member 2110. In other examples, the helical windings may increase in pitch in some areas along the length of the first core member 2110 and may also decrease in pitch in some other areas along the length of the first core member 2110. Such configurations can be utilized to tune the flexibility or stiffness of one or more designated areas or regions along the length of the guide wire assembly 2100. In other words, a first region having a first average pitch is associated with a first stiffness and a second region having a second average pitch is associated with a second stiffness.

Turning now to FIG. 4, a guide wire system 3000 having a dual helical core member configuration is illustrated. As shown, the guide wire system 3000 includes a guide wire assembly 3100 and a controller 1200 electrically coupled to the guide wire assembly 3100. As discussed above, in some examples, the controller 1200 includes or is otherwise electrically coupled with a power source that is configured to deliver or otherwise induce a current through a guide wire assembly, such as guide wire assembly 3100. As shown in FIG. 4, the controller 1200 is coupled to the guide wire assembly 3100 via leads 1302 and 1304.

A cross-sectional view of the guide wire assembly 3100 is illustrated as including a first core member 3110 and a second core member 3120 coupled to one another at a joint 3130. Like the guide wire assembly 1100, the guide wire assembly 3100 is generally cylindrically shaped having a generally circular cross-section and includes an elongate shaft having a proximal end 3102 and a distal end 3104. As shown, the joint 3130 is proximate the distal end 3104 of the guide wire assembly 3100. In various examples, joint 3130 is constructed in the same or similar manner as joint 1130 discussed above.

The first and second core members 3110 and 3120 are each generally similar to the first and second core members 1110 and 1120 of the guide wire assembly 1100 in that the first and second core members 3110 and 3120 each include a body having a proximal and distal end. Likewise, each of the first and second core members 3110 and 3120 include an intermediate portion situated between the proximal and distal ends of the core member.

The first and second core members 3110 and 3120 are each helically wound about a central axis of the guide wire assembly 3100. In various examples, the first and second core members 3110 and 3120 are each predisposed to maintain their respective helical winding configuration when their temperatures are elevated above a designated or critical temperature. For instance, like the second core member 2120 discussed above, in some examples, the first and second core members 3110 and 3120 are each configured to stiffen or lose flexibility but adopt or otherwise maintain their helically wound configuration when their temperatures are elevated above a designated or critical temperature.

In some examples, one of the first and second core members 3110 and 3120 may be configured to maintain its configuration and flexibility or stiffness despite being elevated above a designated or critical temperature. For instance, similar to the discussion above with regard to the second core member 2120, in some examples, one of the first and second core members 3110 and 3120 may be heat treated such that it is not predisposed to stiffen or lose flexibility as its temperature is elevated, but rather generally maintains its stiffness or flexibility across an operating temperature range. In some other examples, one of the first and second core members 3110 and 3120 may alternatively be formed from a non-phase changeable alloy or material that is not operable to change in flexibility as its temperature is elevated.

In a manner similar to or the same as that discussed above regarding guide wire assembly 1100, an insulative layer is disposed about each of the first and second core members 3110 and 3120. Specifically, as shown in FIG. 4, a first insulative layer 3150 is disposed about the first core member 3110, and a second insulative layer 3140 is disposed about the second core member 3120. Though not illustrated in FIG. 4, those of skill should appreciate that in addition to any insulative layers that are individually disposed about the core members 3120 and 3110, an insulative layer may additionally be disposed about the plurality of core members 3110 and 3120.

In various examples, the insulative layers 3140 and 3150 are constructed and disposed about their respective core members as discussed herein.

While the first and second core members 3110 and 3120 are illustrated in FIG. 4 as having generally constant helical windings, it should be appreciated that the first and second core members 3110 and 3120 may be wound about the longitudinal axis of the guide wire assembly 3100 with helical windings that vary in pitch along the length of the guide wire assembly 3100. As mentioned above, in some examples, the helical windings may generally progressively increase (or alternatively decrease) in pitch. In other examples, the helical windings may increase in pitch in some areas and may also decrease in pitch in some other areas. Such configurations can be utilized to tune the flexibility or stiffness of one or more designated areas or regions along the length of the guide wire assembly 3100.

The inventive scope of the concepts addressed in this disclosure has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the inventive scope.

1.-15. (canceled)

16. A medical system comprising:
a guide wire assembly including:
a guide wire member including an alloy and having a flexibility that is configured to change when exposed to an electrical current; and
an insulation material surrounding at least a portion of the guide wire member; and
a controller electrically coupled to the guide wire assembly and configured to cause an electrical current to be selectively supplied to the guide wire assembly such that the flexibility of the guide wire assembly changes in response to an exposure to the electrical current.

17. The medical system of claim 16, wherein the alloy comprises a phase-changeable alloy.

18. The medical system of claim 16, wherein the alloy comprises nitinol.

19. The medical system of claim 16, wherein the guide wire member comprises a first core member and a second core member coupled to the first core member the first core member comprising the alloy such that the guide wire member is configured to change its flexibility when exposed to the electrical current, wherein the first and second core members are coupled to one another at respective first ends of the first and second core members, and wherein respective second ends of the first and second core members are coupled with the controller.

20. The medical system of claim 19, wherein one or more of the first and second core members extend generally linearly along a longitudinal axis of the guide wire assembly when exposed to electrical current.

21. The medical system of claim 19, wherein the first and second core members are aligned parallel to one another.

22. The medical system of claim 19, wherein the second core member is helically coiled about the first core member.

23. The medical system of claim 19, wherein the first and second core members are each helically wound about a longitudinal axis of the guide wire assembly.

24. The medical system of claim 19, wherein the first core member and the second core member are formed from different materials.

25. The medical system of claim 19, wherein the first core member and the second core member are formed from different alloys.

26. The medical system of claim 16, wherein the guide wire assembly varies in flexibility to allow it to function for at least two of the following guide wire purposes: tracking, deployment, and cannulation.

27. The medical system of claim 16, wherein the controller is operable to cause a current to flow through a first portion of the guide wire member and wherein the insulation material surrounds the first portion.

28. A medical system comprising:
a guide wire assembly configured to transition between a first configuration and a second configuration, wherein a flexibility of the guide wire assembly in the first configuration exceeds the flexibility of the guide wire assembly in the second configuration, the guide wire assembly including:
a guide wire member comprising an alloy; and
an insulation material surrounding at least a portion of the guide wire member; and
a controller electrically coupled to the guide wire assembly and configured to cause an electrical current to be selectively supplied to the guide wire assembly to cause the guide wire assembly to transition between the first and second configurations.

29. The medical system of claim 28, wherein the guide wire member comprises a first core member and a second core member coupled to the first core member the first core member comprising the alloy such that the guide wire member is configured to change its flexibility when exposed to the electrical current, wherein the first and second core members are coupled to one another at respective first ends of the first and second core members, and wherein respective second ends of the first and second core members are coupled with the controller.

30. The medical system of claim 29, wherein one or more of the first and second core members extend generally linearly along a longitudinal axis of the guide wire assembly when exposed to electrical current.

31. The medical system of claim 29, wherein the first and second core members are aligned parallel to one another.

32. The medical system of claim 29, wherein the second core member is helically coiled about the first core member.

33. The medical system of claim 29, wherein the first and second core members are each helically wound about a longitudinal axis of the guide wire assembly.

34. The medical system of claim 29, wherein the first core member and the second core member are formed from different materials.

35. The medical system of claim 29, wherein the first core member and the second core member are formed from different alloys.
